(19) United States
(12) Patent Application Publication
(10) Pub. No.: US 2011/0236908 A1
(43) Pub. Date: Sep. 29, 2011

(54) TOTAL PLASMA FVII/FVIIA LEVELS AS INDICATORS OF PRE-ECLAMPSIA OF PREGNANT FEMALES

(75) Inventors: Bashir A. Lwaleed, Hampshire (GB); Luci Maria Sant Ana Dusse, Pampulha Belo Horizonte (BR)

(73) Assignee: UNIVERSITY OF SOUTHAMPTON, Southampton (GB)

(21) Appl. No.: 12/994,451

(22) PCT Filed: May 22, 2009
(86) PCT No.: PCT/GB2009/050557

§ 371 Date: Jun. 14, 2011

(30) Foreign Application Priority Data
May 23, 2008 (GB) 0809376.7

(51) Int. Cl. G01N 33/573; C12Q 1/37

(52) U.S. Cl. 435/7.4; 435/23

(57) ABSTRACT

Raised total plasma FVII protein, including activated FVII (FVIIa) in pregnant females, compared with total plasma FVII protein in normal pregnancy, has been found to be an indicator of the pregnancy complication of pre-eclampsia.

### FIELD OF THE INVENTION

The present invention relates to use of plasma Factor VII (FVII) as a diagnostic marker for pre-eclampsia (P-EC) in pregnant females. More particularly, the invention relates to determining whether total plasma FVII protein, including detection of activated FVII (FVIIa), is raised compared with that found in normal pregnancy. Such raised FVII in plasma samples from females with P-EC has been found using a simple immunoassay which detects FVII and FVIIa whether or not complexed with Tissue Factor.

### BACKGROUND TO THE INVENTION

It is well-established that during the course of normal pregnancy a local hypercoagulable state is essential for placental homeostasis. This, however, becomes more profound with development of the complication of pre-eclampsia, a complex multisystem disorder of unknown etiology. Small placental thrombi are frequently observed in women with P-EC suggesting that, in addition to the thrombotic nature of the placental vasculature, pre-disposing factors to thrombosis may cause or contribute to the development of this condition (Bremme and Blomback (1996) Gynecol. Obstet. Invest. 41, 20-26). Thrombus formation may compromise placental prefusion and fetal growth development, possibly leading to intrauterine death. Hence, there has been growing interest in intravascular coagulation in accounting for many of the features of P-EC (see for example Howie et al. (1976) Lancet 2, 323-325; Howie, (1977) Clin. Obstet. Gynaecol. 4, 595-611; Schjetlin et al. (1999) Acta Obstet. Gynecol. Scand. 78, 191-197). Nevertheless, diagnosis of P-EC is currently generally based on clinical observation of hypertension, proteinuria and other related symptoms, although such diagnosis has deficiencies and does not always provide a reliable guide for timing of delivery to ensure the best chance of a viable fetus.

While others have previously looked at coagulation factors in relation to the development of P-EC, Øien et al. found using a one stage clotting assay significantly lower Factor VII activity in pregnant women with severe P-EC in the third trimester compared to a normal pregnant group (Br. J. Obstet. Gynaecol. (1985) 92, 511-517) and such association between severe P-EC and reduced Factor VII activity has also been suggested by Sandset al. (Thrombosis Res. (1989) 55, 665-670). Others have reported lower or no significant difference in Factor VII levels in pregnant women with P-EC compared to normal pregnancy controls (Djeimi\check{s}et al. (1997) Coll. Antropol. 1, study a FVII ELISA assay, this was different from the FVII/FVIIa assay of the inventors; it employed polyclonal antibodies for both antigen capture and detection. Moreover, lower FVII was only found in plasma samples from women with P-EC and fetal growth retardation. Djelmi\check{s} et al. employed a Radonja Diagnostics reagent. However, the sensitivity and precise specificity of that reagent is not known by the inventors and again may differ from the FVII ELISA technique they employed, which as noted above significantly detects both FVII and FVIIa antigen, whether or not complexed to Tissue Factor.

In contrast, Bremme and Blombäck (Gynecol. Obstet. Invest. Ibid) reported that plasma Factor VII levels were raised in women with an earlier diagnosis of P-EC at 6-15 months after birth and with blood sampling performed at least 1 month after the end of lactation, but notably high levels of FVII by ELISA assay were only found in 3 out of 13 plasma ‘leftover’ samples tested by that assay from women with earlier diagnosis of severe P-EC as part of a more extensive study primarily aimed at understanding risk factors for development of essential hypertension after P-EC and no measure was made of FVII levels during pregnancy. Moreover, the exclusion criteria employed for both the mild and severe P-EC groups studied did not include such conditions as kidney disease, glucose intolerance, hereditary hyperglycemia, hereditary hypertension and borderline hypertension, or smoking (although there is known to be a link between smoking and coagulation activation). Furthermore, homeostatic markers are known to normalize 5-6 weeks postpartum in both normal pregnant and P-EC women. For instance, von Willebrand Factor has been reported to increase in women with mild and severe P-EC, but to return to normal levels by 5 weeks post-partum (Deng et al. (1994) Obstet. Gynecol. 84, 941-945). According to information from the Royal College of Obstetricians and Gynaecologists, UK, most women with P-EC will return to normality within 6 weeks following delivery. Hence, nothing of clinical relevance concerning development of P-EC can be extrapolated from the FVII results of Bremme and Blomback obtained at a far longer time point after delivery.

Factor VII binds to tissue factor (TF) following vascular wall damage and is activated by protease cleavage to FVIIa. In the presence of Ca²⁺ and lipid, TF: FVII: FVIIa complex activates both FIX and FX leading eventually to thrombin generation and fibrin clot formation. TF is inhibited by tissue factor pathway inhibitor (TFPI).

By using antibodies which bind to FVII such that both FVII and FVIIa are captured and detected, either alone or complexed with TF, to determine total FVII protein in plasma samples, the inventors have now found that total plasma FVII protein is significantly elevated in the blood of third trimester pregnant women with P-EC compared with total plasma FVII protein in normal pregnancy controls and in healthy non-pregnant age-matched females. In contrast, comparable changes in FVIIa, TF or TFPI were not observed. Hence, raised total FVII protein level, above that to be expected in normal pregnancy, is now proposed as a useful marker of P-EC.

### SUMMARY OF THE INVENTION

The present invention thus provides a method of diagnosing P-EC in a pregnant female which comprises determining whether total FVII protein (Factor VII and Factor VIIa) is raised in a blood or plasma sample from said female compared with the level of total FVII protein in comparable samples from age-matched pregnant women without P-EC. Using plasma samples, such measurement of total FVII protein in the third trimester was found to give a high sensitivity and specificity for severe P-EC individuals diagnosed by hypertension and proteinuria. Severe P-EC was defined by diastolic blood pressure >110 mmHg at admission, or >90 mmHg on two or more consecutive occasions, 4 hours apart; and proteinuria (either ≧300 mg protein per day or a urinary protein/creatinine ratio ≧30 mg/mmol) occurring after the 20th week of pregnancy. In contrast, normal pregnant women employed as controls had a systolic/diastolic blood pressure below 120/80 mmHg and no history of hypertension or proteinuria.

### BRIEF DESCRIPTION OF THE FIGURES

FIG. 1: The distribution of total plasma FVII levels (ng/ml) in the groups studied: group 1: pregnant women diagnosed conventionally as having severe P-EC; group 2: age-matched normal pregnant women (normal preg) and group 3: age-matched non-pregnant healthy women (non-preg). Horizontal lines indicate the median value for each group.

FIG. 2: The area under the curve (AUC) of the relative operating characteristic (ROC) curve for plasma FVII levels in women with P-EC vs healthy non-pregnant women.

FIG. 3: The AUC of the ROC curve for plasma FVII levels in women with P-EC vs normal pregnant women.

### DETAILED DESCRIPTION

As indicated above, the present invention relies on measuring total FVII protein (FVII and FVIIa) in plasma samples of pregnant women. Such measurement my preferably take the form of an immunoassay employing a capture antibody and detection system for bound antigen such that there is determination of both FVII and FVIIa, whether complexed or uncomplexed to TF. Such an immunoassay may take the form of a conventional enzyme-linked immunosorbent (ELISA) assay, for example employing the IMUBIND® Factor VII ELISA kit (American Diagnostica Inc., Stamford, Conn., USA) wherein anti-FVII/FVIIa polyclonal antibody is used as the capture antibody and a biotinylated monoclonal antibody which binds FVII and FVIIa is employed for detection of bound antigen. It will be appreciated that equivalent immunoassays may be employed with, for example, a different labelled monoclonal antibody which recognises the same FVII epitope and for a different capture antibody (monoclonal or polyclonal). Indeed any immunoassay may be employed provided it gives the same detection specificity for total FVII antigen. Conveniently, captured antigen may be detected, using a biotinylated antibody and addition of enzyme—labelled biotin specific binding partner, e.g. horseradish peroxidase—labelled streptavidin as when using the above-noted kit. When using such enzyme-labelled antibody for detection, sensitivity may be increased by the use of a sulphuric acid stop solution, e.g. 0.5N sulphuric acid as illustrated by the exemplification. However, other detection systems for bound antigen may be employed comprising labelled secondary antibody. Any type of detectable label conventionally employed in the immunoassay art might be used. By way of examples, fluorescent labels and chemiluminescent labels might alternatively be employed.

It may be chosen to use determination of FVII protein as above as a preliminary screen for P-EC followed by measurement of blood pressure and urinary protein or urinary protein/creatine ratio. Determination of plasma FVII protein may also be used to confirm diagnosis of P-EC indicated by hypertension and/or proteinuria determination.

The following example illustrates the invention and was carried out employing a kit as noted above for ELISA determination of total Factor VII protein and other ELISA methods for determining FVIIa, TF and TFPI.

### Example

### Materials and Methods

### Subjects

Ethical committee approval was granted for the study by the Southampton and South West Hants Local Research Ethics Committee. Informed consent has been sought from all participants and the relevant clinical details for each subject have been recorded. A total of 57 age matched women were studied. These included healthy non-pregnant women (n=22; mean age=29±6.04), normal pregnant women (n=15; mean age=30.4±5.04), and women with P-EC (n=20; mean age=29.1±5.59), at the third trimester. As noted above, severe P-EC was defined by diastolic blood pressure >110 mmHg at admission, or >90 mmHg on two or more consecutive occasions, 4 hours apart; and proteinuria (either ≧300 mg protein per day or an urinary protein/creatinine ratio ≧30 mg/mmol) occurring after 20th week of pregnancy. The healthy non-pregnant or normal pregnant women (control groups) had systolic/diastolic blood pressure below 120/80 mmHg and no history of hypertension or proteinuria. Exclusion criteria common for the three groups were chronic hypertension, coagulation disturbance or haemostatic abnormalities, cardiovascular diseases, cancer, diabetes, renal and hepatic diseases, anticoagulant or corticosteroids therapy, and smoking. None of the women had hypertension in the reproductive years or P-EC during previous pregnancies.

### Sample Collection

Five mls of venous blood were collected; using a 21-gauge needle, into vacutainer tubes (Becton-Dickinson) containing 3.8% trisodium citrate anticoagulant solution in the proportion of 9 volumes of blood to 1 volume of anticoagulant solution. Following centrifugation of whole blood in 1.5 ml Eppendorf tubes according to the manufacturer instructions for each assay (American Diagnostica Inc., Stamford, Conn., USA); plasma samples were immediately isolated and stored at −70° C. for batch-wise analysis. For each assay, a previously unthawed aliquot was used.

### Measurements of the TF-Dependent Factors

Commercially available enzyme-linked immunosorbent assay (ELISA) kits (IMUBIND®; American Diagnostica Inc., Stamford, Conn., USA) were used according to the manufacturer's instructions to measure FVII, FVIIa, TF and TFPI in blood plasma. The assays which were employed are further described below.

### Plasma—TF

TF levels were determined by IMUBIND® TF ELISA assay. Test samples are added into duplicate wells of a microtitre plate pre-coated with capture antibody (murine anti-human TF monoclonal antibody). TF is then detected using a biotinylated antibody fragment that specifically recognizes bound TF. The subsequent binding of streptavidin-conjugated horseradish peroxidase (HRP) completes the formation of the antibody enzyme detection complex. The addition of TMB substrate and its subsequent reaction with HRP produces a blue colour solution with bound enzyme. The reaction is stopped with sulphuric acid, and the absorbance read at 450 nm. The values are then calculated automatically from a standard curve after subtraction of background values from blank wells. The IMUBIND® TF ELISA assay recognises TF-apo, TF and TF:FVII complexes. No significant interference from other blood coagulation factors or inhibitors of procoagulant activity was observed for the assay. The lower detection limit is approximatelyl 10 pg/ml. The intra and inter assay coefficient of variations were 4.5% and 7.5%, respectively (American Diagnostica Inc., Stamford, Conn., USA).

### Plasma—FVII

FVII levels were measured using the IMUBIND® Factor VII ELISA Kit. In brief, the IMUBIND FVII ELISA employs an anti-FVII/FVIIa polyclonal antibody as the capture antibody. Diluted plasma samples containing FVII/FVIIa are incubated in micro-test wells precoated with the anti-FVII/FVIIa capture antibody. After washing with buffer, the immunocaptured FVII is detected using a biotinylated anti-FVII monoclonal antibody. Addition of horseradish peroxidase labeled streptavidin (HRP) completes the formation of the antibody enzyme detection complex. The addition of TMB substrate and its subsequent reaction with HRP produces a blue coloured solution. The reaction is stopped and the sensitivity is increased by addition of a 0.5 M sulfuric acid stop solution, yielding a yellow color. FVII levels are determined by measuring the absorbance of the diluted sample solution at 450 nm and comparing to those of a standard curve generated using known amounts of FVII. This assay measures native FVII and FVIIa as well as recombinant human FVIIa (NovoSeven®). The ELISA also measures FVII and FVIIa complexed with TF (TF/FVII, TF/FVIIa). The working range of the assay is between 1-50 ng/ml FVII (American Diagnostica Inc., Stamford, Conn., USA).

### Plasma—FVIIa

FVIIa levels were quantified by the IMUBIND® Factor VIIa ELISA assay. The assay employs a biotinylated enzyme inhibitor of FVIIa and an anti-FVII/FVIIa monoclonal antibody as a capture antibody. The samples containing FVIIa are incubated with the biotinylated inhibitor, which covalently attaches to the FVIIa but not FVII. The samples are then added to micro-test wells pre-coated with FVIIa capture antibody. FVIIa is detected by binding of streptavidin conjugated horseradish peroxidase (HRP) conjugate to the immunocaptured FVIIa/biotinylated inhibitor complex. The addition of TMB substrate and its subsequent reaction with HRP provides a blue colour in the presence of bound FVIIa. Sensitivity is increased by the addition of a 0.5N sulphuric acid stop solution, yielding a yellow colour. FVIIa levels are determined by measuring sample solution absorbance at 450 nm and comparison against a standard curve developed using known amounts of FVIIa. The assay recognizes native and recombinant human FVIIa and FVIIa/TF complexes. No significant amounts of FVII are detected in the assay. FVII does not auto-activate to FVIIa during performance of this assay. The lower limit of detection was determined by adding 2 standard deviations to the mean optical density value (n=15) for the FVII deficient plasma (and calculating the corresponding concentration from the standard curve. The level of FVIIa in pooled normal plasma was found to be 5+2 ng/ml (American Diagnostica Inc., Stamford, Conn., USA).

### Total Plasma—TFPI

Total TFPI levels were assessed suing the IMUBIND® Total TFPI ELISA. In brief, the test samples are added into duplicate wells of a microtitre plate pre-coated with capture antibody. TFPI is then detected using a biotinylated monoclonal antibody specific for the TFPI Kunitz domain 1. The subsequent binding of streptavidin conjugated horseradish peroxidase completes the formation of the antibody enzyme detection complex. The addition of TMB substrate and its subsequent reaction with HRP provides a blue colour with bound TFPI. The reaction is stopped with sulphuric acid, and the absorbance read at 450 nm. The values are than calculated automatically from a standard curve after subtraction of background values from blank wells. The Imubind® Total TFPI ELISA assay recognises native and recombinant human TFPI in complex and truncated forms. No significant cross-reactivity or interference from other coagulation factors has been observed for the assay (American Diagnostica Inc., Stamford, Conn., USA). The lower limit of detection for the assay was 0.18 ng/ml. The intra and inter assay coefficient of variations for 5 ng/ml TFPI were 6.5% and 5.5%, respectively (American Diagnostica Inc., Stamford, Conn., USA).

### Statistical Analysis

Data were included in a database and analyzed by Sigma Stat software system version 1.0. Data were not normally distributed, and summary statistics were expressed as medians and interquartile ranges (IQR). Differences between two or more groups were assessed by either Mann-Whitney U-Test or Kruskal-Wallis One-Way Analysis by Ranks or Dunn's method. P<0.05 was considered to be statistically significant. Reliability measures were assessed using the following conventional formulas:
Sensitivity=true positive/true positive+false negative
Specificity=true negative/true negative+false positive
Positive predictive value=true positive/true positive+false positive
Negative predictive value=true negative/true negative+false negative

The sensitivity and specificity were also determined by measuring the Area Under the Curve (AUC) and the 95% confidence interval (CI) of the Relative Operating Characteristic (ROC) curve.

### Results

In summary, total plasma FVII protein levels were significantly elevated in women with P-EC compared to total plasma FVII protein levels in the healthy, non-pregnant or the normal pregnant women groups. However, there were no comparable changes in the other haemostatic factors studied, i.e., FVIIa, TF and TFPI.

### Plasma FVII

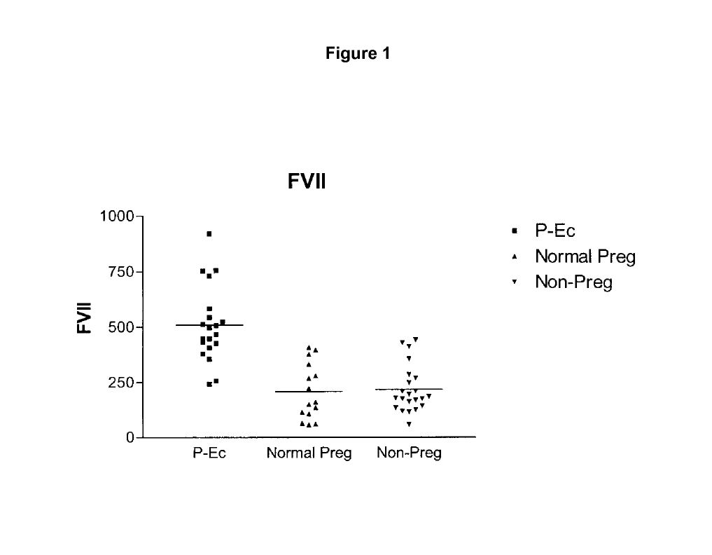

Plasma FVII levels showed higher median and IQR range for women with P-EC (n=20; Median=482.63; IQR=418.28-563.70) when compared to the healthy non pregnant (n=22; Median=182.88; IQR=143.5-270.52) or the normal pregnant group (n=15; Median=160.88; IQR=107.14-333.8). This was statistically significant against the healthy non-pregnant group (P<0.001) or the normal pregnant women group (P<0.001). The distribution of the results is shown in FIG. 1.

### Plasma—FVIIa

The median and IQR for plasma FVIIa levels did not differ among the three groups studied; women with P-EC (n=20; Median=4.34; IQR=3.28-7.16), healthy non pregnant (n=22; Median=4.51; IQR=3.45-6.31) or the normal pregnant group (n=15; Median=4.72; IQR=3.17-5.31).

### Plasma—TF

Plasma TF levels showed no meaningful differences when the three groups where tested against each other. The median and IQR for these groups were women with P-EC (n=20; Median=24.85; IQR=4.0-45.71), healthy non-pregnant group (n=22; Median=16.5; IQR=11.63-26.71) and normal pregnant women group (n=15; Median=11.54; IQR=6.62-17.04).

### Plasma—TFPI

The P-EC and the healthy non pregnant women groups showed slightly higher median and IQR range for plasma TFPI levels when compared to the normal pregnant women group. However, there was no significant difference in plasma TFPI levels between the three groups. The median and IQR range for the three groups were: women with P-EC (n=20; Median=36.17; IQR=28.76-44.61), healthy non pregnant women (n=22; Median=36.79; IQR=30.36-45.48) and normal pregnant women (n=15; Median=30.4; IQR=24.

### Reliability of FVII Levels in Detecting P-EC

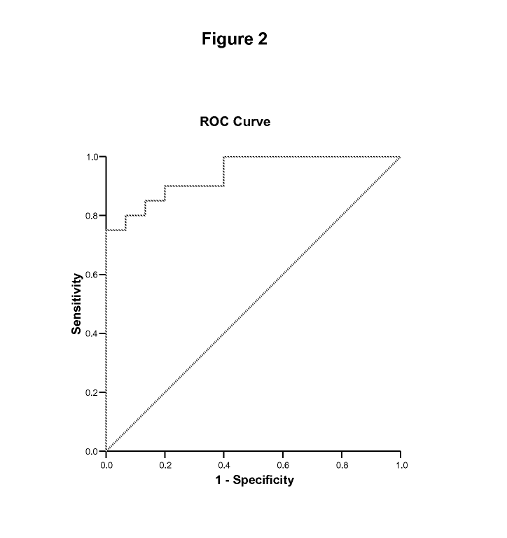

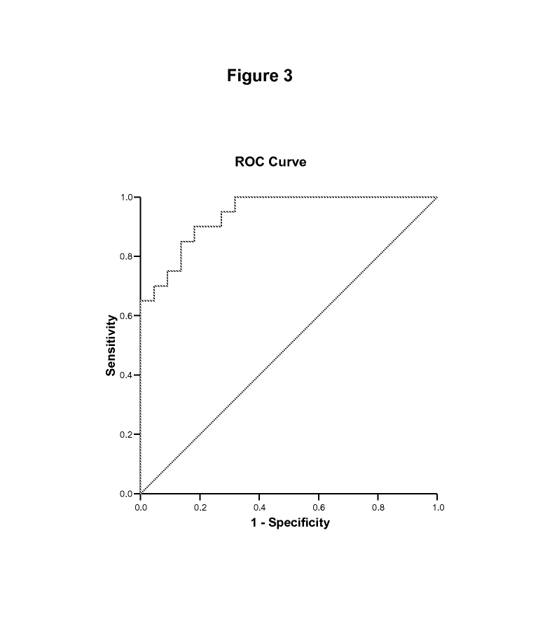

Plasma FVII levels can distinguish women with P-EC from healthy non-pregnant women or normal pregnant women, at the third trimester, with high sensitivity (90%) and specificity (80%). Other reliability measures include true positive (86%); false positive (14%); true negative (86%); false negative (14%). Thus, the positive and negative predictive values were 86%. Using the ROC curve, plasma FVII levels, again, showed sensitivity and specificity in detecting P-EC. The AUC and the 95% confidence interval (CI) for the relative operating characteristic (ROC) curve against the healthy non-pregnant women or normal pregnant women groups were (0.94; 90% CI=0.87-1.0; P<0.001; FIG. 2) and (0.941; 90% CI=0.88-1.0; P<0.001; FIG. 3), respectively. These are summarized in Table 1.

TABLE 1

| | Area | 95% Confidence | | |
|---|---|---|---|---|
| | Under | Interval | | |
| | the Curve | Lower | Upper | P-Value* |
| P-EC vs Healthy pregnant | 0.940 | 0.87 | 1.0 | P < 0.001 |
| women | | | | |
| P-EC vs Normal pregnant | 0.941 | 0.88 | 1.0 | P < 0.001 |
| women | | | | |

P < 0.05 was considered to be statistically significant

1. A method of diagnosing pre-eclampsia (P-EC) in a pregnant female which comprises determining whether total FVII protein (Factor VII and Factor Vila) is raised in a blood or plasma sample from said female compared with the level of total FVII protein in comparable samples from age-matched pregnant women without P-EC.

2. The method of claim 1 wherein said sample is obtained in the third trimester of pregnancy.

3. The method of claim 1 wherein said sample is a plasma sample.

4. The method of claim 3 which is an immunoassay which detects both Factor VII and Factor VIIa, whether or not complexed with tissue factor.

5. The method of claim 4 which is an enzyme-linked immunosorbent assay (ELISA).

6. The method of claim 1 which further comprises measurement of blood pressure and urinary protein or urinary creatine/protein ratio.

7. The method of claim 1 which is used to confirm diagnosis of P-EC indicated by hypertension and/or proteinuria determination.

8. The method of claim 2 wherein said sample is a plasma sample.

9. The method of claim 5 which further comprises measurement of blood pressure and urinary protein or urinary creatine/protein ratio.

10. The method of claim 5 which is used to confirm diagnosis of P-ED indicated by hypertension and/or proteinuria determination.
